(19) United States
(12) Patent Application Publication
(10) Pub. No.: US 2024/0379202 A1
(43) Pub. Date: Nov. 14, 2024

(54) SYSTEM AND METHODS FOR OUTPUTTING HIGHLY VARIABLE CLINICAL STATEMENTS

(71) Applicant: Unified Health, LLC, Eagle, ID (US)

(72) Inventors: Troy Darmody, Eagle, ID (US); Robb Rich, Green Bay, WI (US); Leonard Jensen, Eagle, ID (US); Thomas Rich, Eagle, ID (US)

(21) Appl. No.: 18/195,079

(22) Filed: May 9, 2023

(51) Int. Cl. G16H 15/00; G06F 40/166; G06F 40/295; G06F 40/40; G16H 10/60

(52) U.S. Cl. CPC G16H 15/00; G06F 40/166; G06F 40/295; G06F 40/40; G16H 10/60

(57) ABSTRACT

This disclosure relates generally to generating reports for patient encounters or visits with a clinician. Disclosed are systems and methods of use thereof regarding generation of variable entries for a clinical or encounter statement. In some embodiments, disclosed methods include preparing a customized user interface; conducting a series of randomization algorithms for randomizing one or more sentence structures of a clinical statement, one or more clinical finding values, and one or more structures of a patient name; and/or one or more structures of a clinician name. The method can also include receiving a first input via the customized user interface; and generating the clinical statement based on the received first input. The clinical statement may include at least one variable entry that corresponds to one or more of the randomized sentence structures resulting from the series of randomization algorithms.

### TECHNICAL FIELD

This disclosure relates generally to the field of practicing medicine and, in particular, reporting generation and processes for patient encounters or visits with a clinician.

### SUMMARY

Disclosed are systems and methods of use thereof regarding generation of variable entries for a clinical or encounter statement. In some embodiments, disclosed methods of generating variable entries in a clinical statement include preparing a customized user interface, the customized user interface corresponding to a clinician role and a patient encounter type, based on the clinical role and the patient encounter type, conducting a first series of randomization algorithms for randomizing one or more sentence structures of the clinical statement, conducting a second series of randomization algorithms for randomizing one or more sentence structures for one or more clinical finding values, conducting a third series of randomization algorithms for randomizing one or more sentence structures of patient information, receiving a first input corresponding to one or more clinical finding values, the one or more clinical finding values input via the customized user interface, generating the clinical statement based on the received first input, the clinical statement including at least one variable entry that corresponds to the randomized sentence structure of one of the clinical finding values, receiving a second input corresponding to an accuracy of the generated clinical statement, and generating a final clinical statement based on the received second input, the final clinical statement including one or more variable entries, where the one or more variable entries can include the at least one variable entry from the clinical statement.

In some embodiments, disclosed methods of providing narrative or entry variations in a clinical statement include conducting a first series of randomization algorithms to prepare a first clinical statement template, receiving a first input corresponding to one or more clinical finding values, generating an encounter statement based on the received first input, the encounter statement including at least one variable narrative, receiving a second input corresponding to an accuracy of the generated encounter statement, and generating a final encounter statement based on the received second input, the final encounter statement including one or more variable narratives, where the one or more variable narratives can include the at least one variable narrative from the encounter statement.

Also disclosed are systems. In some embodiments, disclosed systems for outputting a clinical statement includes a sentence content structure randomization algorithm/module/instruction/phrase engine/spinner for randomly selecting a sentence content structure for a sentence of the clinical statement, a clinical value randomization algorithm/module/processor for randomly selecting a sentence content structure for a sentence of a clinical value, a patient name randomization algorithm/module for randomly selecting a sentence content structure for a sentence of patient information, and a clinician role randomization algorithm/module for randomly selecting a sentence content structure for a sentence of a clinician role.

Other aspects of the disclosed subject matter, as well as features and advantages of various aspects of the disclosed subject matter, should be apparent to those of ordinary skill in the art through consideration of the ensuing description, the accompanying drawings, and the appended claims.

### BRIEF DESCRIPTION OF THE DRAWINGS

In the drawings:

FIG. 1 illustrates one example of a user interface for generating clinical statements, according to the present disclosure;

FIGS. 2-5 illustrate close-up views of various segments or portions of the user interface of FIG. 1;

FIGS. 6-9 illustrate flowcharts of example methods for generating variable clinical statements, according to the present disclosure;

FIG. 10 illustrates a block diagram of a system, according to the present disclosure; and

FIG. 11 illustrates a block diagram of a system, according to the present disclosure.

### DETAILED DESCRIPTION

A clinical statement is a report or note detailing an interaction between a clinician (e.g., a physician, a nurse, a tech, etc.) and a patient. Each clinical statement includes various entries regarding the interaction between the clinician and the patient. Typically, clinical statements include entries and details regarding the encounter itself, such as the date, time, location, and parties present (e.g., names, ages, identifiers, etc.). Additionally, clinical statements include substantive details regarding the patient's condition, treatments, complications, condition timeline, and potential action items for the future (such as at a future interaction). These clinical statements are usually put together or prepared by the clinician present in the interaction, visit, or encounter. Based on the discussion during the interaction, the clinician makes notes and observations regarding the interaction, the patient and any conditions they might have, potential treatments, and any other relevant or pertinent information. These notes and observations are compiled into the clinical statement as a final report for the interaction, visit, or encounter.

Often, more than one clinician is present or assisting in the interaction. The observations of every present clinician will be aggregated or included in the clinical statement for the interaction, visit, or encounter. The notes and observations from any clinician may generally fall under the same repeated categories. For example, pediatric clinicians may make repeated observations regarding the age and development of a patient, as well as parental information. As another example, oncology clinicians may make repeated observations regarding the success of a chemotherapy or other treatment.

Another consideration is the duplication of effort confronted by medical professionals and clinicians each time they create a patient report. Such reports necessarily begin with the same or similar information, including the patient's name, age, gender, underlying condition, and related information. It is an obvious waste of time for clinicians to have to regenerate that information in every clinical statement. It is also essential that most relevant and specific information is recorded.

Despite the repetitive nature of many of these interactions, visits, or encounters, regulations require clinicians to prepare fresh clinical statements for each interaction. That is, clinicians are not allowed to simply “cut-and-paste” observations or notes from one clinical statement into another clinical statement, regardless of how closely the observations overlap. Several regulations and standards in the United States discourage or prohibit the practice of copying and pasting previous notes into subsequent patient documentation. For example, under the Health Insurance Portability and Accountability Act (HIPAA), copying and pasting previous notes can potentially compromise the accuracy and integrity of a patient's medical record, which may violate HIPAA requirements. Centers for Medicare & Medicaid Services (CMS) Guidelines provide guidance on the use of cloning (copying and pasting) in Electronic

Health Records (EHRs) as part of its efforts to prevent fraud, waste, and abuse. CMS states that cloning of documentation is inappropriate and that each patient encounter should be documented separately to ensure the medical necessity of the services provided. The Joint Commission, which accredits and certifies health care organizations in the United States, has also issued statements discouraging the use of copy-pasting in clinical documentation. The commission emphasizes the importance of accurate and complete patient records to ensure patient safety and quality of care. The American Health Information Management Association (AHIMA) has published guidelines on copy-pasting in health records, stressing the importance of accurate and reliable documentation. They advise against using copy-pasting without proper modification and validation, as it may lead to inaccurate or incomplete information in the patient's health record.

Because of the limitations on copying and pasting, generation of these clinical statements can take time and focus of the clinician away from other tasks, such as treating patients.

Disclosed are systems and methods of use thereof regarding generation of variable entries for a clinical statement or encounter statement. A clinical statement may sometimes be referred to herein as “a patient encounter statement.” In some embodiments, disclosed methods of generating variable entries in a clinical statement include preparing a customized user interface, the customized user interface corresponding to a clinician role and a patient encounter type, based on the clinical role and the patient encounter type, conducting a first series of randomization algorithms for randomizing one or more sentence structures of the clinical statement, conducting a second series of randomization algorithms for randomizing one or more structures for one or more clinical finding values, and conducting a third series of randomization algorithms for randomizing one or more structures of patient identification or patient name structure. The method also includes receiving a first input corresponding to one or more clinical finding values, the one or more clinical finding values input via the customized user interface, receiving an input associated with a patient's name, and generating the clinical statement based on the received first input and the received patient's name. The generated clinical statement includes at least one variable entry that corresponds to the randomized sentence structure of one of the clinical finding values. The method can also include receiving a second input corresponding to an accuracy of the generated clinical statement, and generating a final clinical statement based on the received second input, the final clinical statement including one or more variable entries, where the one or more variable entries can include the at least one variable entry from the clinical statement.

In some embodiments, disclosed methods of providing narrative or entry variations in a clinical statement include conducting a first series of randomization algorithms to prepare a first clinical statement template, receiving a first input corresponding to one or more clinical finding values, generating an encounter statement based on the received first input, the encounter statement including at least one variable narrative, receiving a second input corresponding to an accuracy of the generated encounter statement, and generating a final encounter statement based on the received second input, the final encounter statement including one or more variable narratives, where the one or more variable narratives can include the at least one variable narrative from the encounter statement.

Also disclosed are systems. In some embodiments, disclosed systems for outputting a clinical statement includes a clinical statement sentence content structure module for storing and randomly selecting a sentence content structure for sentence(s) of the clinical statement, a clinical finding value structure module for storing and randomly selecting a content structure for a clinical value, a patient name structure module for storing and randomly selecting a structure for a patient name, and a clinician name structure module for storing and randomly selecting a clinician name structure for a clinician name.

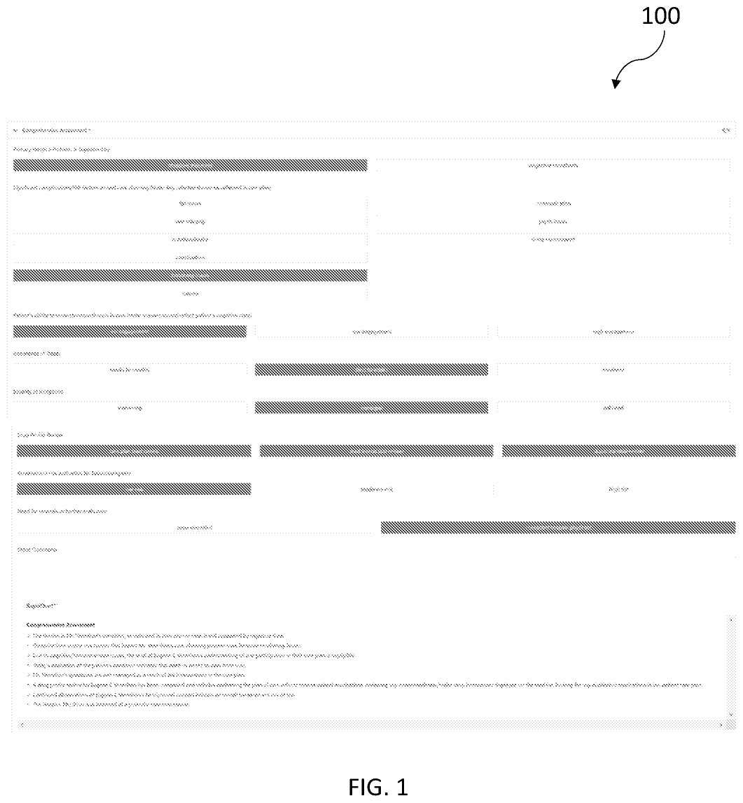

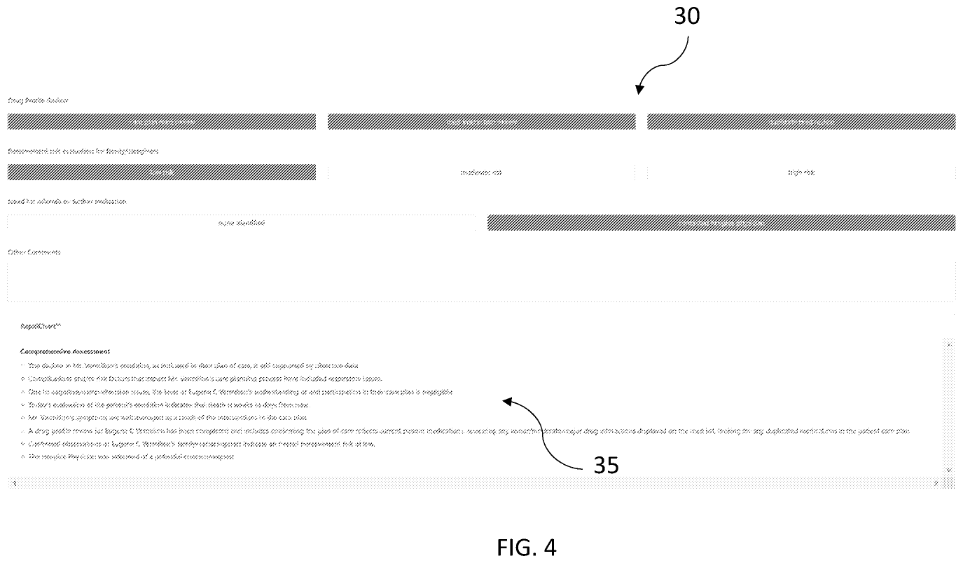

FIG. 1 illustrates one example of a user interface 100 for generating clinical statements, according to the present disclosure. The user interface 100 may be customized or tailored to a particular role of a clinician, a particular type of patient interaction or encounter, and/or combinations thereof. In some embodiments, the user interface 100 receives various inputs (e.g., discrete inputs) regarding, for example, objective or subjective measures, complications or risk factors around care planning, a patient's condition, drugs or other treatment factors, and/or comments or concerns of the clinician. The various inputs can be received in or by the user interface 100 via buttons, icons, drop-down menus, checkboxes, multiple choice, text fields, or any suitable input format. As discussed with respect to FIG. 4, the user interface 100 also provides or displays an assessment based on the received inputs.

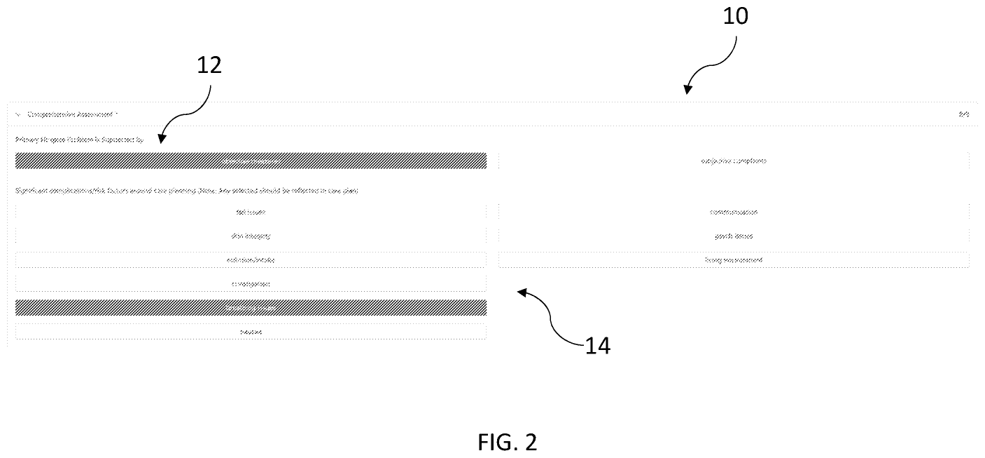

FIGS. 2-4 illustrate close-up views of various segments or portions of the user interface 100 of FIG. 1. Specifically, FIG. 2 illustrates a measure and complication segment 10 of the user interface 100. The segment 10 allows inputs relating to, for example, objective and/or subjective measures 12. As illustrated, objective measures have been selected or input in the segment 10. The segment 10 also allows for inputs relating to complications or risk factors 14 that may impact care planning for a patient. As illustrated, the complications or risk factors 14 include fall issues, skin integrity, nutrition/intake, constipation, breathing issues, and/or nausea, which may correlate to objective measures 12. The complications or risk factors 14 may also include communication, psychiatric/psychological issues, and/or living environment factors, which may correlate to subjective measures 12.

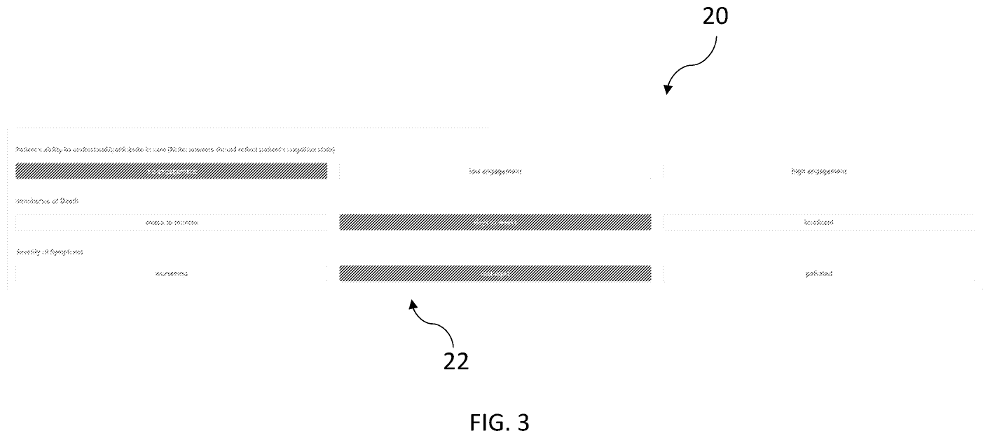

FIG. 3 illustrates a patient capacity and engagement segment 20 of the user interface 100. The segment 20 allows for or receives inputs relating to a patient's capacity to engage in and understand a care plan (e.g., a plan of treatment or maintenance). For example, the segment 20 allows for inputs regarding a level of engagement for the patient (e.g., not engaged, low engagement, high engagement, etc.), an imminence of death for the patient, and/or a severity of symptoms for the patient. As with segment 10, the inputs of segment 20 can be correlated to objective and/or subjective measures 12 (see FIG. 2).

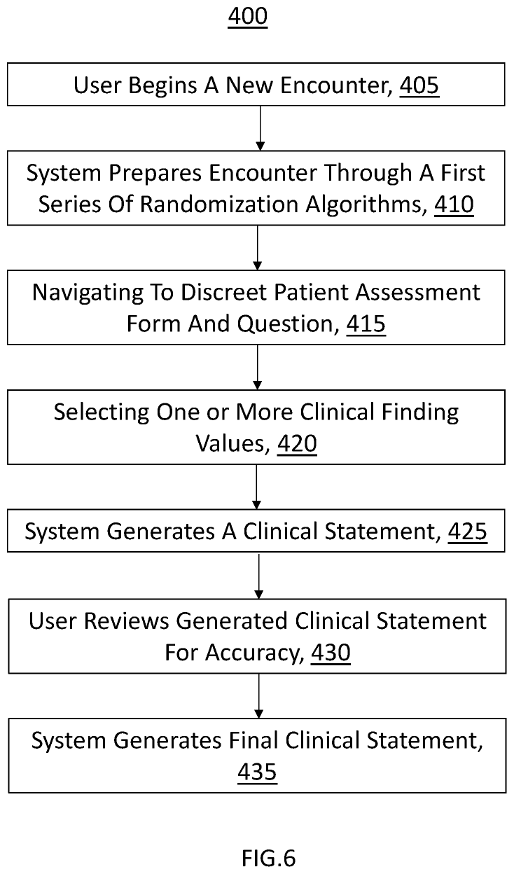

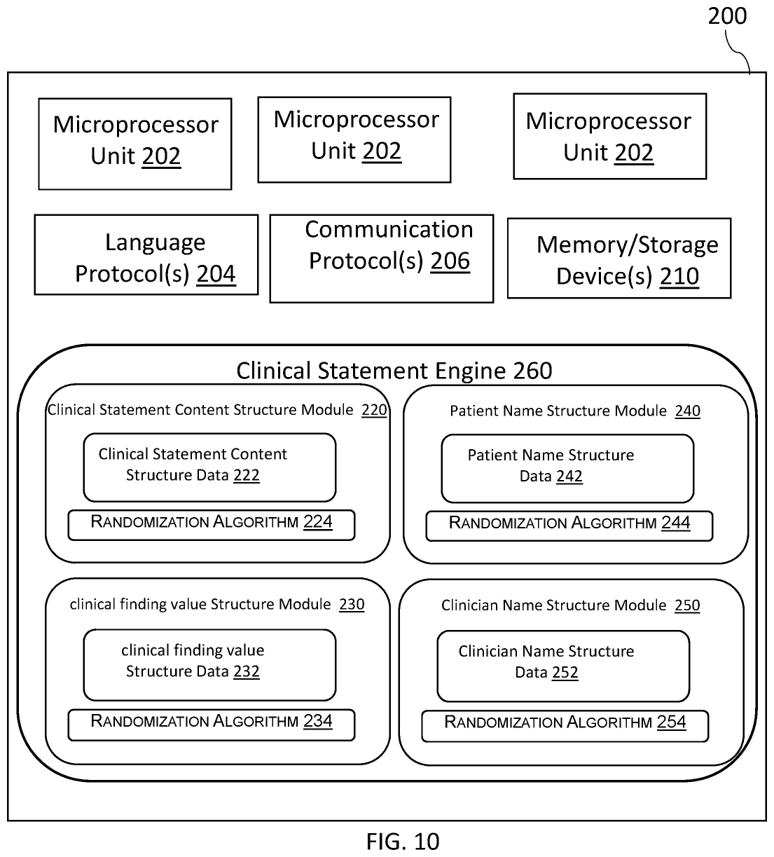

FIG. 4 illustrates a drug profile and extra factors segment 30 of the user interface 100. The segment 30 allows for or receives inputs relating to a drug profile for a patient, a risk evaluation for the patient's family or caregiver, a referral, and comments of the clinician seeing the patient. The segment 30 also provides or displays a generated clinical statement or narrative 35. The clinical statement 35 is generated from the discrete inputs received in each segment 10, 20, 30. More specifically, the clinical statement 35 is a randomized clinical statement arrangement and is generated through a computer-varied clinical statement engine 260, discussed more fully below with respect to FIGS. 6-9, and illustrated in FIG. 10.

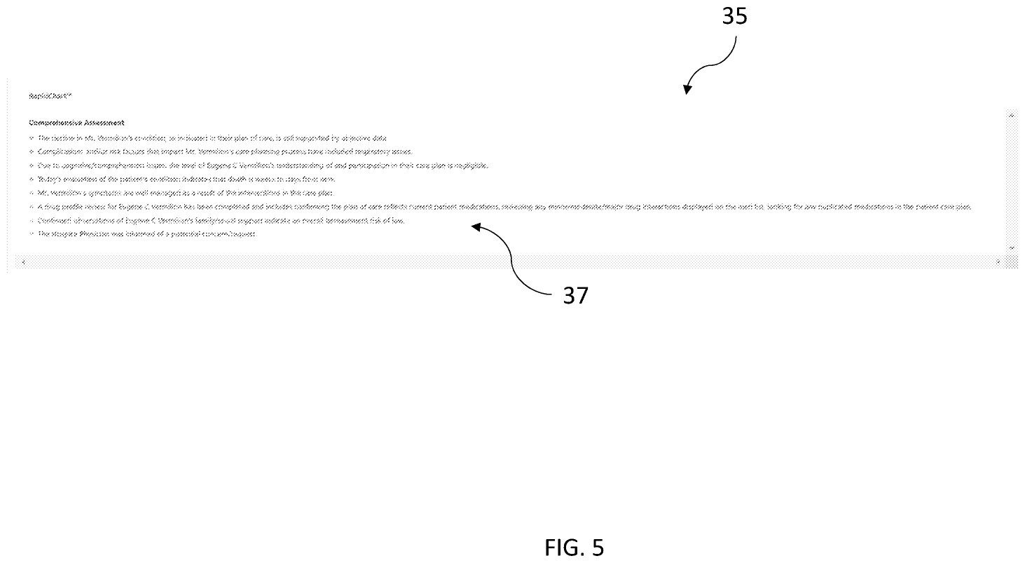

FIG. 5 illustrates the generated clinical statement or narrative 35 from the drug profile and extra factors segment 30. Specifically, the clinical statement 35 includes a plurality of entries 37. The plurality of entries 37 are automatically generated based on (i) the inputs received in each segment 10, 20, 30, (ii) the clinician role, (iii) the type of patient interaction or encounter, (iv) a series of randomization algorithms, (v) the input patient identifier or name, (vi) the input clinician identifier or name, and (vii) combinations thereof. The clinical statement 35 is generated from the various “button push” inputs from segments 10, 20, 30. Each of the various inputs from segments 10, 20, 30 correspond to clinical findings or values. Over the course of a modest number of “button push” clinical findings (e.g., from each input received by the system via segments 10, 20, 30), the clinical statement or narrative 35 output becomes extremely improbable to ever be recreated through the same series of button clicks. That is, each entry of the plurality of entries 37 is unique and has an extremely low probability of ever being repeated. This high degree of narrative output variability is advantageous as compliance regulations state that clinical statements should never be repeated or “cut and paste.” In combination with variance in the patient name structure, clinician name structure, etc., the degree of narrative output variability increases further.

FIGS. 6-9 illustrate flowcharts of example methods for generating variable clinical statements, according to the present disclosure. For example, FIG. 6 illustrates an example method 400 of generating a clinical statement. The method 400 includes a user beginning a new encounter, at 405. In some embodiments, the new encounter is commenced via a user interface, such as user interface 100 of FIGS. 1-5, which may be customized for the user. For example, the user may be a clinician, such as a nurse, a physician, a pharmacist, a phlebotomist, or any other person working in a clinic, hospital, etc. The system, such as the system 200 of FIG. 10, prepares the encounter through a first series of randomization algorithms, at 410. In some embodiments, the first series of randomization algorithms are for randomizing one or more sentence structures for corresponding inputs or values, such as patient information, patient conditions, treatment plans, etc. In other configurations, the system performs the first series of randomization algorithms at a later stage.

The method 400 additionally includes a user/clinician navigating to a discreet patient assessment form and questionnaire, at 415. The patient assessment form and questionnaire may be provided to the clinician via the user interface, another clinician, or the patient. The patient assessment form and questionnaire may be selected by the system based on one of more of the following initial inputs by the clinician: (i) role of the clinician, (ii) type of assessment to be conducted, (iii) identify of the clinician, and/or (iv) identify of the patient. The patient assessment form and questionnaire may include patient information, such as age, sex/gender, name, identifying information, birthdate, family information, employment information, and/or other relevant patient information. The patient assessment form and questionnaire may also include patient condition and/or treatment information.

The method 400 may further include selecting one or more clinical finding values, at 420. In some embodiments, the clinical finding values may correspond to the patient condition and/or treatment information. In some embodiments, the clinical finding values may correspond to complications or risk factors for the patient. For example, the clinical finding values may correspond to the complications or risk factor inputs from the measure and complication segment 10 of the user interface 100 (see FIG. 2).

The method 400 also includes a system, such as the system 200 of FIG. 10, generating a clinical statement, at 425. The clinical statement may be the clinical statement 35 illustrated in FIGS. 4-5 and may be produced and/or displayed by the user interface. For example, the clinical statement may include a plurality of entries (e.g., entries 37) that correspond to the various inputs received by the system. Each entry may be randomly generated from the first series of randomization algorithms that randomized one or more sentence structures for corresponding inputs or values, such as patient information, patient conditions, treatment plans, etc. Each clinical statement produced by the system will be unique to satisfy compliance regulations that clinical notes should never be repeated or “cut-and-paste.” The method 400 also includes review of the generated clinical statement for accuracy, at 430, and generation of a final clinical statement, at 435.

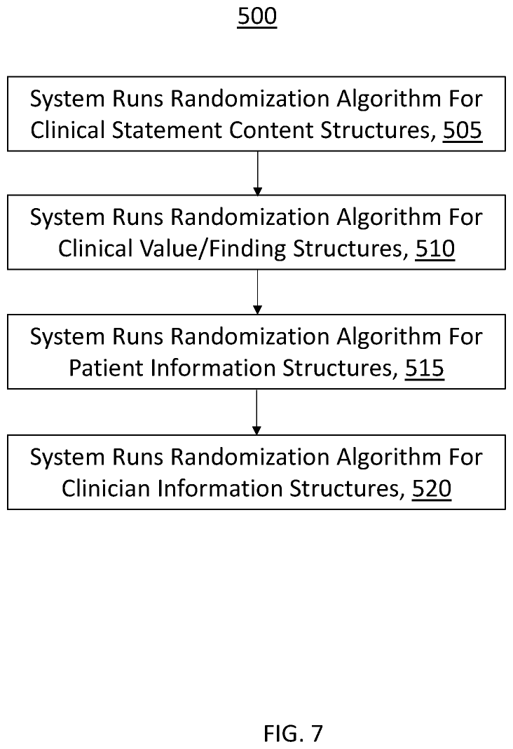

FIG. 7 illustrates another example method 500 of generating a clinical statement. The method 500 includes, at 505, a system (such as system 200 from FIG. 10) running a randomization algorithm for clinical statement content structures. That is, the system will run a randomization algorithm to produce variable sentence structures for each entry 37 in a clinical statement 35. The method 500 also includes the system running a randomization algorithm for clinical values or findings structures, at 510. The method 500 also includes the system running a randomization algorithm for patient name or identification structures, at 515. The method 500 further includes the system running a randomization algorithm for clinician name or identification structures, at 520. When the system receives an input corresponding to clinical findings, patient information, and/or clinician information, the system outputs an entry for a clinical statement. Each entry output by the system is variable and unique, such that the same output will not be provided twice.

As a non-limiting example, when the system receives an input corresponding to a clinical finding that the patient is having “breathing issues,” the system will output a corresponding entry for the clinical statement. The entry will have a randomized sentence structure selected by, for example, a clinical statement content structure module (220 in FIG. 10). Exemplary sentence structures for a clinical finding with respect to “breathing issues” could include any suitable sentence structures. Non-limiting examples include:
<<Patient identifier>>is experiencing<<clinical finding>>
<<Patient identifier>>has<<clinical finding>>
<<Patient identifier>>reports<<clinical finding>>
<<Patient identifier>>complains of<<clinical finding>>
<<Patient identifier>>describes<<clinical finding>>
<<Clinical finding>>are reported by<<Patient identifier>>
   The sentence structures include placeholders for additional inputs for a patient identifier structure or patient name structure, and/or a clinical finding value structure. Each of these placeholders may be independently selected by additional randomization algorithms.

The entry will have a randomized value for the clinical finding value structure selected by, for example, a clinical finding value structure module (230 in FIG. 10). This module may store and randomly select various structures related to a clinical finding value. For example, where the clinical finding value is “breathing issues,” the randomization algorithm for clinical finding value structure may cause the system to output one or more of the following clinical finding value structures: issues with breathing; breathing issues; breathing-related issues; etc. As another non-limiting example, when the system receives an input corresponding to a clinical finding value that the patient is “struggling with nutrition,” the system may output one or more of the following entries to insert into the randomly generated sentence structure that contains a placeholder for the clinical finding for “struggling with nutrition”: malnourished; low calorie intake; poor quality nutrition; poor nutrition intake; nutrient deficient; etc.

Similarly, the entry will have a randomized value for the patient identifier structure or patient name structure selected by, for example, a patient name structure module (240 in FIG. 10). The randomization algorithm for the patient name structure may output one or more of the following patient name structure entries to be inserted for the patient identifier or patient name, where the input patient name is “Jane Anne Doe”: “Jane,” “Ms. Doe,” “Jane Doe,” “Jane A. Doe,” “J. Doe,” “J. A. Doe,” “Ms. J. Doe,” etc.

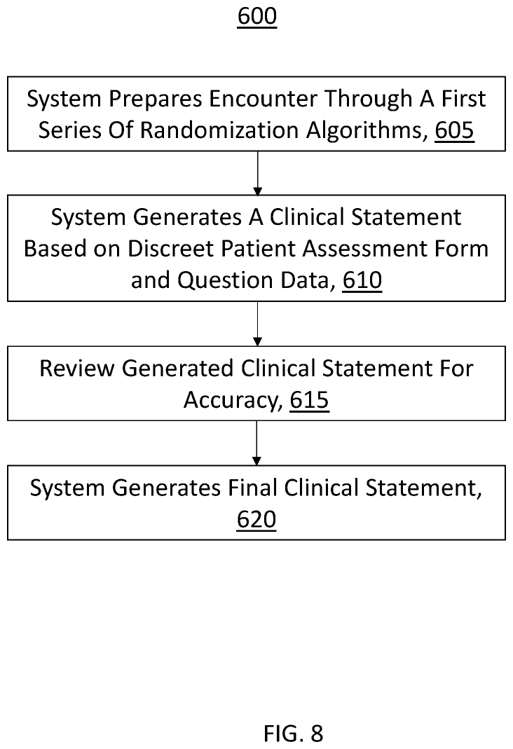

FIG. 8 illustrates another example method 600 of generating a clinical statement. The method 600 includes, at 605, a system (such as system 200 from FIG. 10), preparing an encounter through a first series of randomization algorithms. In some embodiments, the system is preparing the encounter through a user interface, such as user interface 100. That is, for example, the system prepares an encounter for a clinician that the clinician can access through the user interface 100. The method 600 may also include the system generating a clinical statement based on discreet patient assessment form and question data, at 610. In some embodiments, the discreet patient assessment form and question data is entered and/or accessible through the user interface. In some embodiments, the discreet patient assessment form and question data corresponds to the clinical values or findings, such as the inputs illustrated in FIGS. 2-3.

The method 600 also includes reviewing the generated clinical statement for accuracy, at 615, and generating a final clinical statement based on the accuracy of the generated clinical statement, at 620. In some embodiments, the generated clinical statement is reviewed by a clinician through the user interface. The clinician may provide an input to the user interface corresponding to the accuracy of the clinical statement. Such input may include a change to the generated clinical statement (e.g., an addition, a deletion, a modification, etc.) and/or approval of the generated clinical statement. The final clinical statement generated by the system at 620 will include or reflect the change to and/or the approval of the clinical statement by the clinician.

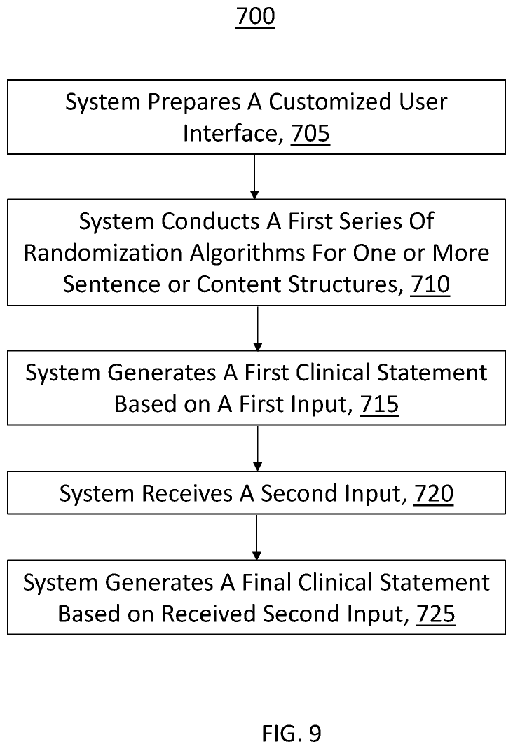

FIG. 9 illustrates another example method 700 of generating a clinical statement. The method 900 includes a system preparing a customized user interface, at 705. As before, the user interface may be user interface 100 of FIG. 1 and may be customized according to (i) the clinician role, (ii) the type of patient interaction or encounter, and (iii) combinations thereof. The method 900 also includes the system conducting a first series of randomization algorithms for one or more sentence or content structure, at 710. For example, the first series of randomization algorithms may be conducted by a clinical statement content structure module (220 in FIG. 10) of a clinical statement engine (260 in FIG. 10). The clinical statement content structure module can randomly select sentence structures for various clinical statements. The system may conduct a randomization algorithm for clinical values or findings structures (such as by a clinical finding value structure module 230 in FIG. 10), a randomization algorithm for patient name structures (such as by a patient name structure module 240 in FIG. 10), and/or a randomization algorithm for clinician name structures (such as by a clinician name structure module 250 in FIG. 10). Such randomization algorithms produce variable sentence structures for each entry in a clinical statement, the entries corresponding to one or more of clinical findings, patient name, and clinician name.

The method 700 further includes the system generating a first clinical statement based on a first input, at 715. In some embodiments, the first input is received via the customized user interface. In some embodiments, the first input corresponds to one or more clinical findings (e.g., breathing issues), patient information, and clinician information. The method 700 includes the system receiving a second input, at 720. In some embodiments, the second input corresponds to an accuracy of the first clinical statement. Such input may include a change to the generated clinical statement (e.g., an addition, a deletion, a modification, etc.) and/or approval of the generated clinical statement. The method 700 also includes generating a final clinical statement based on the received second input, at 725, such that the final clinical statement is an accurate reporting of a patient interaction, visit, or encounter.

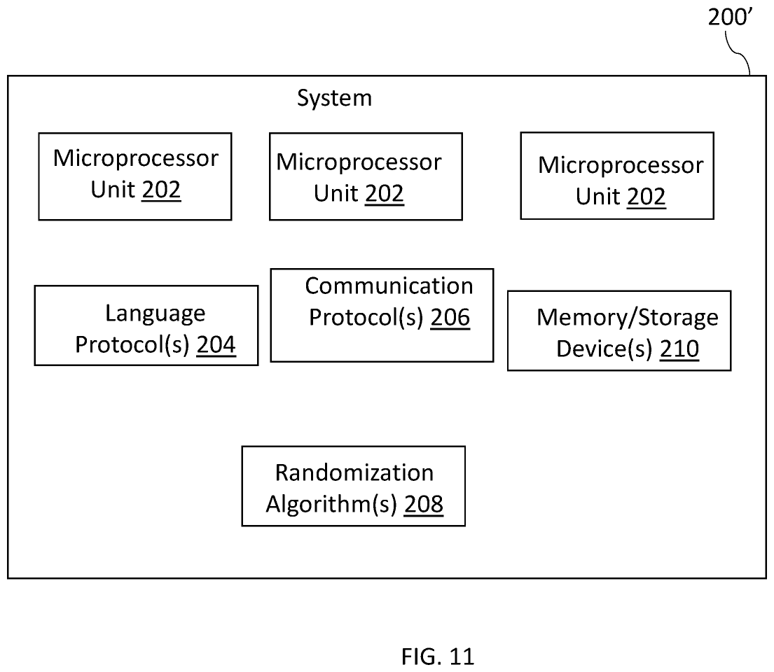

FIGS. 10 and 11 illustrate block diagrams of configurations of a system 200 and 200′, respectively, according to the present disclosure. In some embodiments, the system 200 includes one or more microprocessor units 202, a plurality of protocols, and one or more memory/storage devices 210. The plurality of protocols may include language protocols 204, communication protocols 206, and/or randomization algorithms 208. Each protocol may allow the system to generate and randomize sentence structures for corresponding entries in a clinical statement. The language and/or communications protocols 204, 206 may further allow the system to process and display a clinical statement to a clinician. In some embodiments, the system is implemented in a computer and the plurality of protocols 204, 206, 208 are readable and executable by the computer. In some embodiments, the plurality of protocols 204, 206, 208 are stored in the memory/storage device 210.

In some configurations, the system 200 includes a clinical statement content structure module 220, which includes clinical statement content structure data 222 and a randomization algorithm 224 or other rules for randomization of the selection from the clinical statement content structure data 222. The clinical statement content structure data 222 may include, for example, a plurality of sentence structures associated with one or more entries 37. A clinical statement may include a plurality of entries 37, wherein each entry represents a particular clinical finding. For each of the entries 37 of the clinical statement 35, the clinical statement content structure module may randomly select a sentence structure for the particular clinical finding. Or, the system may use a particular set of rules to select a sentence structure for each particular clinical finding. Within the sentence structure, there may be a plurality of placeholders for data, such as a placeholder for a clinical finding value structure, a patient name structure, a clinician name structure, etc.

The system 200 may also include a clinical finding value structure module 230, which includes clinical finding value structure data 232 and a randomization algorithm 234 or other rules for randomization of the selection from the clinical finding value structure data 232. The system 200 may also include a patient name structure module 240, which includes patient name structure data 242 and a randomization algorithm 234 or other rules for randomization of the selection from the patient name structure data 242. Optionally, the system 200 may also include a clinician name structure module 250, which includes clinician name structure data 252 and a randomization algorithm 254 or other rules for randomization of the selection from the patient name structure data 252.

### Additional Terms and Definitions

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It should also be noted that some of the embodiments disclosed herein may have been disclosed in relation to a particular application with respect to clinical statements; however, other applications are also contemplated.

In one embodiment, the terms “about” and “approximately” refer to numerical parameters within 10% of the indicated range. The terms “a,” “an,” “the,” and similar referents used in the context of describing the embodiments of the present disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the embodiments of the present disclosure and does not pose a limitation on the scope of the present disclosure. No language in the specification should be construed as indicating any non-claimed element essential to the practice of the embodiments of the present disclosure.

The terms “patient name” and “clinician name” can include the legal name of the patient or clinician, and/or any other information relating to the patient or clinician (such as role, etc.).

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the author(s) of this disclosure for carrying out the embodiments disclosed herein. Of course, variations on these described embodiments will become apparent to those of ordinary skill in the art upon reading the foregoing description. The author(s) expects skilled artisans to employ such variations as appropriate, and the author(s) intends for the embodiments of the present disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of this disclosure so claimed are inherently or expressly described and enabled herein.

Although this disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. A method of generating variable entries in a clinical statement comprising:
preparing a customized user interface, the customized user interface corresponding to a clinician role and a patient encounter type;
based on the clinical role and the patient encounter type, conducting a first series of randomization algorithms for randomizing a sentence structure of the clinical statement and generating a randomized sentence structure;
conducting a second series of randomization algorithms for randomizing a structure of clinical finding value and generating a randomized clinical finding value structure;
conducting a third series of randomization algorithms for randomizing a structure of a patient name and generating a randomized patient name structure;
receiving a first input corresponding to a clinical finding value, the clinical finding value input via the customized user interface;
receiving a second input corresponding to a patient name;
generating the clinical statement based on the received first input and the received second input, the clinical statement including at least one variable entry that corresponds to the randomized sentence structure, randomized clinical finding value structure, and randomized patient name structure;
receiving a third input corresponding to an accuracy of the generated clinical statement; and
generating a final clinical statement based on the received third input.

2. The method of claim 1, wherein generating the clinical statement comprises concatenating results from the first series of randomization algorithms.

3. The method of claim 1, further comprising conducting a fourth series of randomization algorithms for randomizing a structure of a clinician name and generating a randomized clinician name structure.

4. A system for outputting a clinical statement comprising:
a sentence content structure randomization algorithm for randomly selecting a sentence content structure for a sentence of the clinical statement;
a clinical value randomization algorithm for randomly selecting a content structure for a clinical value;
a patient name randomization algorithm for randomly selecting a content structure for a patient name; and
a clinician name randomization algorithm for randomly selecting a content structure for a clinician name.

5. The system of claim 4, further comprising a graphical user interface for generating the clinical statement, the graphical user interface corresponding to a clinician role and a type of patient interaction.

6. The system of claim 4, further comprising a concatenation algorithm for aggregating outputs from the sentence content structure randomization algorithm, the clinical value randomization algorithm, the patient name randomization algorithm, and the clinician name randomization algorithm.

7. The system of claim 6, wherein the concatenation algorithm generates a first clinical statement from the aggregated outputs.

8. The system of claim 7, wherein an accuracy of the first clinical statement is determined through a graphical user interface corresponding to a clinician role and patient information.

9. The system of claim 8, wherein the concatenation algorithm generates a second clinical statement based on the accuracy of the first clinical statement.

10. A method of providing narrative variations in a clinical statement comprising:
conducting a first randomization algorithm to prepare a clinical statement template;
receiving a first input corresponding to one or more clinical finding values;
conducting a second randomization algorithm to generate one or more random clinical finding value structures;
generating an encounter statement based on the clinical statement template, the received first input and the generated one or more random clinical finding value structures, the encounter statement including at least one variable narrative;
receiving a second input corresponding to an accuracy of the generated encounter statement; and
generating a final encounter statement based on the received second input, the final encounter statement including one or more variable narratives.

11. The method of claim 10, wherein generating an encounter statement comprises generating more than one encounter statement, each generated encounter statement based on the received first input.

12. A system for outputting a clinical statement comprising:
a sentence content structure module for storing and randomly selecting a sentence content structure for a sentence of the clinical statement, the sentence content structure containing a placeholder for a clinical finding value structure and a placeholder for a patient name structure;
a clinical finding value structure module for storing and randomly selecting a clinical finding value structure; and
a patient name structure module for storing and randomly selecting a patient name structure.

13. The system of claim 12, further comprising a clinical statement engine for concatenating the sentence content structure with the randomly selected clinical finding value structure of the clinical finding value structure module and the randomly selected patient name structure module of the patient name structure module to output a clinical statement, the clinical statement comprised of the sentence content structure randomly selected by the sentence content structure module, with the placeholder for insertion of a clinical finding value structure filled by the randomly selected clinical finding value structure and the placeholder for insertion of a patient name structure filled by the randomly selected patient name structure.

14. The system of claim 13, further comprising a clinician name structure module for storing and randomly selecting a content structure for a clinician name.

15. The system of claim 14, wherein the sentence content structure contains a placeholder for a clinician name structure.

16. The system of claim 15, wherein the clinical statement is further comprised of the sentence content structure randomly selected by the sentence content structure module, with the placeholder for insertion of a clinician filled by the randomly selected clinician name structure.

17. The system of claim 13, wherein the clinical finding value structure is separately randomly generated and wherein the patient name structure is separately randomly generated.
